SEQUENCE LISTING (31 sequences)

SEQ ID NO: 1 - protein, 118 aa, Callithrix jacchus
QVQLVQSGAEVKKPGASVKVSCKASGYTFTSYAISWARQPPGQGLEWMGAFDPEYGSTTYAQKFQGRVTMTADTSTSTAYMELSSLRPEDTAVYYCARDVNFGNYFDYWGQGTLVTVS

SEQ ID NO: 2 - protein, 330 aa, Homo sapiens [sequence omitted]

SEQ ID NO: 3 - protein, 108 aa, Callithrix jacchus
ELVMTQSPATLSLSPGERATVSCRAGQSVSYYLAWYQQKPGQAPRLLIYGASTRATGIPARFSGSRSGTDFTLTISSLEPEDFAVYYCQQYSSWPPTFGQGTKLEIKR

SEQ ID NO: 4 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 449 aa, Artificial Sequence
  AB138 heavy chain sequence
QVQLVQSGAEVKKPGASVKVSCKASGYTFTSYAISWARQPPGQGLEWMGAFDPEYGSTTYAQKFQGRVTMTADTSTSTAYMELSSLRPEDTAVYYCARDVNFGNYFDYWGQGTLVTVSSASTKNPDVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  AB138 light chain sequence
ELVMTQSPATLSLSPGERATVSCRAGQSVSYYLAWYQQKPGQAPRLLIYGASTRATGIPARFSGSRSGTDFTLTISSLEPEDFAVYYCQQYSSWPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 107 aa, Artificial Sequence
  Domain antibody acceptor sequence
DIQMTQSPSSLSASGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 8 - protein, 105 aa, Aotus nancymaae
LSLPVTPGEPASISCRSSQSLLHSNGNTYLRWYLQKPGKPPQLLVYKVSNRASGVPDRFSGSGSGTDFTLKISRVEAEDVGVYYCMSYLQAPMYTFGQGTKVEIK

SEQ ID NO: 9 - protein, 99 aa, Aotus nancymaae
SSLSASVGDRVTITCHASQSISNWLAWYQQKPGKVPKLLIYKASTLQSGVPSRFSGSGSGTDFTLTISSLQPEDVATYYCQKYDSSPWTFGKGTKLEIK

SEQ ID NO: 10 - protein, 99 aa, Aotus nancymaae
SSLSASVGDRVTITCHTSQSISNWLAWYQQKPGKVPKLLIYAASTLQSGVPSRFSGSGSGTDFTLIISSLQPEDVATYYCQKYDSSPYTFGQGTKVEIK

SEQ ID NO: 11 - protein, 106 aa, Aotus nancymaae
LSLPITLGESASISCRSSQSLLDSDYGFTYLDWYLQKPGQSPQVLIYAASNRASGVPDRFSGSGADTDFTLKISRVEAEDVGVYYCMQSKELPPFTFGPGTKVEIK

SEQ ID NO: 12 - protein, 99 aa, Aotus nancymaae
SSLSASVGDRVTITCRASQDIYNFLAWYQQKPGKTPRLLIYTSSNLQAGIPSRFSGSGSGTDYTLTISSLQPDDFATYYCQHGYNTPFTFGPGTKVEIK

SEQ ID NO: 13 - protein, 99 aa, Aotus nancymaae
SSLSASVGDKVTITCRASQGISKYLAWYQQKPGKAPKPLIYYASSLQSGIPSRFSGSGSGADYTLTISSLQPEDFATYYCQQYNSFPLTFGQGTKVEIK

SEQ ID NO: 14 - protein, 99 aa, Aotus nancymaae
SSLSASVGDKVTITCRASQDINKYLVWYQQKPGKAPKPLIYYASFLQGGVPSSFSGSGSGADYTLTISSLQPEDFATYYCQQYNSFPRTFGQGTRIEIK

SEQ ID NO: 15 - protein, 104 aa, Aotus nancymaae
LSLPVTPGEPASISCRSSQSLLHSNGSTYLYWFLQKPGQPPQLLIYRVSNRASGVPDRFSGSGSGTDFTLKISRVEAEDVGVYYCMNYLQPPYTFGQGTKVEIK

SEQ ID NO: 16 - protein, 99 aa, Aotus nancymaae
SSLSAPVGDRVTITCHASQSISNWLAWYQQKPGKVPKLLIYKASTLQSGVPSRFSGSGSGTDFTLTISSLQPEDVATYYRQKYDSSPYTFGQGTKVEIK

SEQ ID NO: 17 - protein, 99 aa, Aotus nancymaae
ATLSLSPKETATLSCRASQSVSSSLAWYQQKPGQAPRLLIYGASTRATGIPARFSGSGSGTDFTLTISSLEPEDFAVYYCQQYSNWPYTFGQGTKVEIK

SEQ ID NO: 18 - protein, 104 aa, Aotus nancymaae
LSLPVTPGEPASIFCRSSQSLLHSNGNTYLSWFLQEPGQSPRRLIYKVSTRGPGVPDRFSGSGAGTDFTLKISRVEAEDVGVYYCLQSTQHPRTFGKGTKLEIK

SEQ ID NO: 19 - protein, 130 aa, Callithrix jacchus
  VARIANT 108: Xaa = Any Amino Acid
ELTLTQSPSSLSASVGDRVTITCRASQDIRGYLAWYQQKPGKSPRLLIYSASTLQTGVPSRFSGSRSGTDYTLTISSLQSEDVATYYCQQHYSTPLTFGQGTKLEIKXAVAAPSVFIFPPSEDQVKSGTA

SEQ ID NO: 20 - protein, 130 aa, Callithrix jacchus
ELVMTQSPATLSLSPKETATLSCRASQSVRSYLAWYQQKPGQAPRLLIYGASTRATGIPARFSGSGYGTDFTLTISSLEPEDFAVYYCQQYSSWYTFGQGTKLEIKRAVAAPTVFIFPTSEDQVKSGTAT

SEQ ID NO: 21 - protein, 130 aa, Callithrix jacchus
ELVMTQSPSSLFASIGDRVTITCRASQNIRSNLAWYQQKPGKTPRLLIYDASSLQPGIPSRFSGSGSGTYYTLTISSLQSDDLATYYCQQGYTTPVTFGQGTKLEIKRAVAAPSVFIFPPSEDQVKSGTA

SEQ ID NO: 22 - protein, 130 aa, Callithrix jacchus
ELVMTQSPATLSLSPGERATVSCRAGQSVSYYLAWYQQKPGQAPRLLIYGASTRATGIPARFSGSRSGTDFTLTISSLEPEDFAVYYCQQYSSWPPTFGQGTKLEIKRAVAAPSVFIFPPSEDQVKSGTA

SEQ ID NO: 23 - protein, 130 aa, Callithrix jacchus
ELVMTQSPATLSLSPKETATLSCRASQSVSSYLAWYQQKPGQAPRLLIYGASTRATGIPARFSGSGSGTDFTLTISRLEPEDFAVYYCQQYSSWPLTFGQGTKLEIKRAVAAPSVFIFPPSEDQVKSGTA

SEQ ID NO: 24 - protein, 130 aa, Callithrix jacchus
ELTLTQSPVTLSLSPKETATLSCRASQSVRSYLAWYQQKPGQAPRLLIYGASTRATGIPARFSGSGSGTDFTLTISSLEPEDFAVYYCQQYSSWYTFGQGTKLEIKRAVAAPSVFIFPPSEDQVKSGTAT

SEQ ID NO: 25 - protein, 107 aa, Artificial Sequence
  Domain antibody acceptor sequence with CDR2 substituted for CDR2 of AAF05517
DIQMTQSPSSLSASGDRVTITCRASQSIDSYLHWYQQKPGKAPKLLIYKVSNRASGVPSRFSGSGSGTDFTLTISSLQPEDFATYYCQQVVWRPFTFGQGTKVEIKR

SEQ ID NO: 26 - protein, 107 aa, Artificial Sequence
  Domain antibody acceptor sequence with CDR2 substituted for CDR2 of AAF055120
DIQMTQSPSSLSASGDRVTITCRASQSIDSYLHWYQQKPGKAPKLLIYAASNRASGVPSRFSGSGSGTDFTLTISSLQPEDFATYYCQQVVWRPFTFGQGTKVEIKR

SEQ ID NO: 27 - protein, 107 aa, Artificial Sequence
  Domain antibody acceptor sequence with CDR2 substituted for CDR2 of AAF055121
DIQMTQSPSSLSASGDRVTITCRASQSIDSYLHWYQQKPGKAPKLLIYTSSNLQAGVPSRFSGSGSGTDFTLTISSLQPEDFATYYCQQVVWRPFTFGQGTKVEIKR

SEQ ID NO: 28 - protein, 107 aa, Artificial Sequence
  Domain antibody acceptor sequence with CDR2 substituted for CDR2 of AAF05523
DIQMTQSPSSLSASGDRVTITCRASQSIDSYLHWYQQKPGKAPKLLIYYASFLQGGVPSRFSGSGSGTDFTLTISSLQPEDFATYYCQQVVWRPFTFGQGTKVEIKR

SEQ ID NO: 29 - protein, 107 aa, Artificial Sequence
  Domain antibody acceptor sequence with CDR2 substituted for CDR2 of AAF05524
DIQMTQSPSSLSASGDRVTITCRASQSIDSYLHWYQQKPGKAPKLLIYRVSNRASGVPSRFSGSGSGTDFTLTISSLQPEDFATYYCQQVVWRPFTFGQGTKVEIKR

SEQ ID NO: 30 - protein, 107 aa, Artificial Sequence
  Domain antibody acceptor sequence with CDR2 substituted for CDR2 of AAF05527
DIQMTQSPSSLSASGDRVTITCRASQSIDSYLHWYQQKPGKAPKLLIYKVSTRGPGVPSRFSGSGSGTDFTLTISSLQPEDFATYYCQQVVWRPFTFGQGTKVEIKR

SEQ ID NO: 31 - protein, 107 aa, Artificial Sequence
  Domain antibody acceptor sequence with CDR2 substituted for CDR2 of AAF05556
DIQMTQSPSSLSASGDRVTITCRASQSIDSYLHWYQQKPGKAPKLLIYDASSLQPGVPSRFSGSGSGTDFTLTISSLQPEDFATYYCQQVVWRPFTFGQGTKVEIKR

(19) United States
(12) Patent Application Publication
(10) Pub. No.: US 2008/0255343 A1
(43) Pub. Date: Oct. 16, 2008

(54) CHIMERIC ANTIBODIES

(75) Inventors: Philip A. JENNINGS, Warrawee (AU); Anthony G. Doyle, Drummoyne (AU); Adam W. Clarke, Russell Lea (AU)

Correspondence Address: MORRISON & FOERSTER LLP, 755 PAGE MILL RD, PALO ALTO, CA, 94304-1018, US

(73) Assignee: Arana Therapeutics Limited, Macquarie Park (AU)

(21) Appl. No.: 12/115,264

(22) Filed: May 5, 2008

Related U.S. Application Data
(62) Division of application No. 11/636,338, filed on Dec. 8, 2006.

(51) Int. Cl. C12P 21/00

(52) U.S. Cl. 530/387.3

(57) ABSTRACT

The present invention provides a chimeric antibody or an antigen-binding portion thereof. The antigen-binding portion comprises at least two complementarity determining regions (CDR) and at least three framework regions, wherein at least one CDR is a New World primate CDR

### FIELD OF THE INVENTION

The present invention relates to a chimeric antibody or antigen-binding portion thereof, wherein the antigen binding portion comprises at least two complementarity determining region (CDR) sequences and at least three framework regions, wherein at least one CDR is a New World primate CDR, and to the use of the antibody or antigen-binding portion thereof in treating diseases or disorders.

### BACKGROUND OF THE INVENTION

Antibodies (immunoglobulins) play an important role in the immune system of a mammal. They are produced by plasma cells which have developed from precursor B cells. Antibodies consist of two identical light polypeptide chains and two identical heavy polypeptide chains which are joined by disulfide bridges. The light chains are referred to as either kappa or lambda light chains and the heavy chains as gamma, mu, delta, alpha or epsilon. Each chain consists of a constant and variable region. The variable region gives the antibody its specificity. Within each variable region are regions of hypervariability or complementarity determining regions (CDRs) which are flanked by more conserved regions referred to as framework regions. Within each variable region are three CDRs and four framework regions.

Antibodies are bifunctional molecules, the N-terminal variable segments from the heavy and light chains associate together in a specific manner to generate a three-dimensional structure with affinity for a particular epitope on the surface of an antigen. The constant region segments are responsible for prolonged serum half-life and the effector functions of the antibody and relate to complement binding, stimulation of phagocytosis, antibody-dependent cellular cytotoxicity and triggering of granulocyte granule release.

The development of hybridoma technology has facilitated the production of monoclonal antibodies of a particular specificity. Typically, such hybridomas are murine hybridomas.

Human/mouse chimeric antibodies have been created in which antibody variable region sequences from the mouse genome are combined with antibody constant region sequences from the human genome. The chimeric antibodies exhibit the binding characteristics of the parental mouse antibody, and the effector functions associated with the human constant region. The antibodies are produced by expression in a host cell, including for example Chinese Hamster Ovary (CHO), NSO myeloma cells, COS cells and SP2 cells.

Such chimeric antibodies have been used in human therapy, however antibodies to these chimeric antibodies have been produced by the human recipient. Such anti-chimeric antibodies are detrimental to continued therapy with chimeric antibodies.

It has been suggested that human monoclonal antibodies are expected to be an improvement over mouse monoclonal antibodies for in vivo human therapy. From work done with antibodies from Old World primates (rhesus monkeys and chimpanzees) it has been postulated that these non-human primate antibodies will be tolerated in humans because they are structurally similar to human antibodies (Ehrlich PH et al., Human and primate monoclonal antibodies for in vivo therapy. Clin Chem. 34:9 pg 1681-1688 (1988)). Furthermore, because human antibodies are non-immunogenic in Rhesus monkeys (Ehrich PH et al., Rhesus monkey responses to multiple injections of human monoclonal antibodies. Hybridoma 1987; 6:151-60), it is likely that the converse is also applicable and primate antibodies will be non-immunogenic in humans. These monoclonal antibodies are secreted by hybridomas constructed by fusing lymphocytes to a human x mouse heteromyeloma.

EP 0 605 442 discloses chimeric antibodies which bind human antigens. These antibodies comprise the whole variable region from an Old World monkey and the constant region of a human or chimpanzee antibody. One of the advantages suggested in this reference for these constructs is the ability to raise antibodies in Old World monkeys to human antigens which are less immunogenic in humans compared with antibodies raised in a mouse host.

New World primates (infraorder-Platyrrhini) comprise at least 53 species commonly divided into two families, the Callithricidae and Cebidae. The Callithricidae consist of marmosets and tamarins. The Cebidae includes the squirrel monkey, titi monkey, spider monkey, woolly monkey, capuchin, uakaris, sakis, night or owl monkey and the howler monkey.

Evolutionarily distant primates, such as New World primates, are not only sufficiently different from humans to allow antibodies against human antigens to be generated, but are sufficiently similar to humans to have antibodies similar to human antibodies so that the host does not generate an anti-antibody immune response when such primate-derived antibodies are introduced into a human.

Previous studies have characterised the expressed immunoglobulin heavy chain repertoire of the Callithrix jacchus marmoset (von Budingen H-C et al., Characterization of the expressed immunoglobulin IGHV repertoire in the New World marmoset Callithrix jacchus. Immunogenetics 2001; 53:557-563). Six IGHV subgroups were identified which showed a high degree of sequence similarity to their human IGHV counterparts. The framework regions were more conserved when compared to the complementarity determining regions (CDRs). The degree of similarity between C. jacchus and human IGHV sequences was less than between non-human Old World primates and humans.

### Domain Antibodies

Domain antibodies (dAb) are functional binding units which can be created using antibody frameworks and correspond to the variable regions of either the heavy (V_{H}) or light (V_{L}) chains of antibodies. Domain antibodies have a molecular weight of approximately 13 kDa, or less than one tenth the size of a full antibody.

Immunoglobulin light chains are referred to as either kappa or lambda light chains and the heavy chains as gamma, mu, delta, alpha or epsilon. The variable region gives the antibody its specificity. Within each variable region are regions of hypervariability, otherwise known as complementarity determining regions (CDRs) which are flanked by more conserved regions referred to as framework regions. Within each light and heavy chain variable region are three CDRs and four framework regions.

In contrast to conventional antibodies, domain antibodies are well expressed in bacterial, yeast and mammalian systems. Their small size allows for higher molar quantities per gram of product, thus providing a significant increase in potency. In addition, domain antibodies can be used as a building block to create therapeutic products such as multiple targeting domain antibodies in which a construct containing two or more variable domains bind to two or more therapeutic targets, or domain antibodies targeted for pulmonary or oral administration.

### SUMMARY OF THE INVENTION

The present inventors have found that New World primates provide a rich source of binding domains for antibodies against a range of antigens including human antigens. Further, due to the similarity of the sequences between human and New World primates it is likely that these New World primate sequences will have relatively low immunogenicity in humans.

In a first aspect the present invention provides a chimeric antibody or an antigen-binding portion thereof, wherein the antigen-binding portion comprises at least two complementarity determining regions (CDR) and at least three framework regions, wherein at least one CDR is a New World primate CDR.

In another aspect the present invention provides a method of producing a chimeric antibody or an antigen-binding portion thereof, the method comprising deleting a CDR from a human antibody variable region comprising at least two CDRs and at least three framework regions and replacing it with a New World primate CDR predicted to be of low immunogenicity to produce a chimeric variable region.

In a related aspect the method further comprises the step of recovering the chimeric variable region.

In yet another aspect the present invention provides a chimeric antibody or an antigen-binding portion thereof produced according to the method of the present invention.

In a further aspect, the invention provides a pharmaceutical composition comprising an effective amount of the antibody or antigen-binding portion thereof according to the present invention, together with a pharmaceutically acceptable excipient or diluent.

In a still further aspect, the invention provides for the use of an antibody or antigen-binding portion thereof of the present invention in a diagnostic application for detecting an antigen associated with a particular disease or disorder.

In another aspect, the present invention provides a method for treating a disease or disorder characterised by human TNF-α activity in a human subject, comprising administering to the subject in need thereof an effective amount of a chimeric antibody as described herein, or a pharmaceutical composition thereof in which the antibody or antigen-binding portion thereof binds TNF-α.

### BRIEF DESCRIPTION OF THE FIGURES

FIG. 1 demonstrates the binding of AB138 to rat MOG present in rat spinal cord lysate (lane 2) and not to CHOK1SV lysate (lane 3). Lane 1 contains molecular weight markers.

FIG. 2 demonstrates the lack of non-specific binding of an anti-TNFα monoclonal antibody to the same sample of rat MOG present in rat spinal cord lysate (lane 2) and CHOK1SV lysate (lane 3). Lane 1 contains molecular weight markers.

FIG. 3 shows the acceptor domain antibody amino acid (SEQ ID NO.: 7) and nucleotide sequence (both strands) (SEQ ID NOs.: 50-51). The restriction digest sites for Kpn I and San DI, which excises a region including the CDR2, is indicated in the figure. CDR2 residues are indicated in underline.

FIG. 4 is a sequence alignment of the domain antibody acceptor sequence (SEQ ID NO.: 7) with a panel of New World primate derived immunoglobulin sequences (SEQ ID NOs.: 8-24, listed sequentially from top to bottom in FIG. 4) performed using AlignX (Vector NTI, Invitrogen, Australia). The CDR2 is highlighted in bold text.

FIG. 5 shows CDR2 substituted domain antibodies binding to TNFα. Grey indicates constructs that are predicted to have lower immunogenicity compared to the acceptor domain antibody (SEQ ID No: 7).

### DETAILED DESCRIPTION OF THE INVENTION

In a first aspect the present invention provides a chimeric antibody or an antigen-binding portion thereof, wherein the antigen-binding portion comprises at least two complementarity determining regions (CDR) and at least three framework regions, wherein at least one CDR is a New World primate CDR.

It is preferred that the antigen binding portion comprises three CDRs and four framework regions. It is also preferred that the antigen-binding portion comprises at least one, and preferably two human CDRs.

In some embodiments of the present invention, the chimeric antibody or antigen-binding portion thereof comprises one New World primate CDR. In other embodiments, the chimeric antibody or antigen-binding portion thereof comprises two New World primate CDRs. In other embodiments CDR2 of the antibody or antigen-binding portion is a New World primate CDR.

In other embodiments of the present invention the at least one New World primate CDR is not from a sequence that binds a target antigen.

In other embodiments of the present invention the framework regions are human sequences. Framework regions that are human sequences include sequences derived from human framework regions, or synthetic sequences based on human framework regions.

It is within the scope of the present invention, that the sequence of the antigen binding portion may be further subject to affinity maturation in order to improve its antigen binding characteristics such as antigen binding or potency.

An increase in binding is demonstrated by a decrease in K_{D} (k_{oƒƒ}/kₒₙ) for the antibody or antigen binding portion thereof. An increase in potency is demonstrated in biological assays. For example, assays that can be used to measure the potency of the antibody or antigen-binding portion thereof include the TNFα-induced L929 cytotoxicity neutralisation assay, IL-12-induced human PHA-activated peripheral blood mononuclear cell (PBMC) proliferation assay, and RANKL mediated osteoclast differentiation of mouse splenocytes (Stern, Proc. Natl. Acad. Sci. USA 87:6808-6812 (1990); Kong, Y-Y. et al. Nature 397:315-323 (1990); Matthews, N. and M. L. Neale in Lymphokines and Interferons, a Practical Approach, 1987, M. J. Clemens, A. G. Morris and A. J. H. Gearing, eds., IRL Press, p. 221).

In a further preferred embodiment at least one framework region is modified to increase binding and/or to reduce predicted immunogenicity in humans.

In another embodiment at least one CDR sequence is modified to increase binding or potency and or to reduce predicted immunogenicity in humans. It is preferred that where at least one CDR sequence which is modified it is not the New World primate CDR. Where two or more New World primate CDRs are present then it is preferred that at least one New World primate CDR is not modified.

In other embodiments of the present invention at least one framework region is modified, in addition to at least one CDR sequence, to increase binding and or to reduce predicted immunogenicity in humans. It is preferred that the at the least one CDR sequence which is modified it is not a New World primate CDR sequence.

In a preferred embodiment the antigen-binding portion is a domain antibody.

In a further embodiment of the present invention, the domain antibody may be multimerised, as for example, hetero- or homodimers (e.g., V_{H}/V_{H}, V_{L}/V_{L} or V_{H}/V_{L}), hetero- or homotrimers (e.g., V_{H}/V_{H}/V_{H}, V_{L}/V_{L}/V_{L}, V_{H}/V_{H}/V_{L} or V_{H}/V_{L}/V_{L}), hetero- or homotetramers (e.g., V_{H}/V_{H}/V_{H}/V_{H}, V_{L}/V_{L}/V_{L}/V_{L}, V_{H}/V_{H}/V_{H}, V_{L}/V_{H}/V_{H}/V_{L}/V_{L} or V_{H}/V_{L}/V_{L}/V_{L}), or higher order hetero- or homomultimers. Multimerisation can increase the strength of antigen binding, wherein the strength of binding is related to the sum of the binding affinities, or part thereof, of the multiple binding sites.

Thus, the invention provides a domain antibody wherein the domain antibody is linked to at least one further domain antibody. Each domain antibody may bind to the same or different antigens.

The domain antibody multimers may further comprise one or more domain antibodies which are linked and wherein each domain antibody binds to a different antigen, multi-specific ligands including so-called “dual-specific ligands”. For example, the dual specific ligands may comprise a pair of V_{H} domains or a pair of V_{L} domains. Such dual-specific ligands are described in WO 2004/003019 (PCT/GB2003/002804) in the name of Domantis Ltd incorporated by reference herein in its entirety.

Preferably, the antibody or antigen-binding portion further comprises a human or non-human primate constant region sequence. Examples of non-human primates include, but are not limited to, chimpanzees, oranguatangs and baboons.

The present invention also provides a method of producing a chimeric antibody or an antigen-binding portion thereof, the method comprising deleting a CDR from a human antibody variable region comprising at least two CDRs and at least three framework regions and replacing it with a New World primate CDR predicted to be of low immunogenicity to produce a chimeric variable region.

In a related aspect the method further comprises the step of recovering the chimeric variable region.

It is preferred that the selected New World primate CDR is CDR2. It is preferred that the CDR2 sequence is selected from KVSNRAS (SEQ ID NO.: 32), RVSNRAS (SEQ ID NO.: 33), KVSTRGP (SEQ ID NO.: 34), AASNRAS (SEQ ID NO.: 35), TSSNLQA (SEQ ID NO.: 36), DASSLQP (SEQ ID NO.: 37) and YASFLQG (SEQ ID NO.: 38). Particularly preferred sequences are KVSNRAS (SEQ ID NO.: 32), AASNRAS (SEQ ID NO.: 35), TSSNLQA (SEQ ID NO.: 36) and KVSTRGP (SEQ ID NO.: 34) due to their predicted lower immunogenicity.

In further embodiments the method further comprises modifying the sequence of the chimeric variable region to increase binding and/or to decrease immunogenicity in humans. It is preferred that the New World primate CDR sequence is not modified. Where two or more New World primate CDR sequences are present then it is preferred that at least one New World primate CDR is not modified.

In other embodiments of the present invention at least one framework region is modified in addition to at least one CDR sequence, to increase binding and or to reduce predicted immunogenicity in humans. It is preferred that the at the least one CDR sequence which is modified it is not a New World primate CDR sequence. The present invention also provides a chimeric antibody or an antigen-binding portion thereof produced by the method of the present invention.

The term “antibody” as used herein, is intended to refer to immunoglobulin molecules comprised of four polypeptide chains, two heavy (H) chains and two light (L) chains inter-connected by disulfide bonds. Each heavy chain is comprised of a heavy chain variable region (HCVR or V_{H}) and a heavy chain constant region. The heavy chain constant region comprises three domains, C_{H}1, C_{H}2 and C_{H}3. Each light chain is comprised of a light chain variable region (LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

The term “antigen-binding portion” of an antibody, as used herein refers to one or more components or derivatives of an immunoglobulin that exhibit the ability to bind to an antigen. It has been shown that the antigen-binding function of an antibody can be performed by fragments of a full length antibody. Examples of binding fragments encompassed within the term “antigen-binding portion” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody; (v) a dAb fragment (Ward et al, 1989, Nature 341:544-546) which consists of a single V_{H} domain, or a V_{L} domain (van den Beuken T et al, 2001, J. Mol. Biol, 310, 591); and (vi) an isolated complementarity determining region (CDR). Furthermore, although the two domains of the Fv fragment, V_{L} and V_{H}, are coded by separate genes, they can be joined, using recombinant methods, by a synthetic linker that enables them to be made as a single protein chain in which the V_{L} and V_{H} regions pair to form monovalent molecules (known as single chain Fv (scFv); (see eg Bird et al., 1988, Science 242:423-426 and Huston et al., 1988 Proc. Natl. Acad. Sci. USA 85:5879-5883). Such single chain Fvs are also intended to be encompassed within the term “antigen-binding portion” of an antibody. Other forms of single chain Fvs and related molecules such as diabodies or triabodies are also encompassed. Diabodies are bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., 1993, Proc. Natl. Acad. Sci. USA, 90:6444-6448; Poljak, R. J., et al., 1994, Structure, 2:1121-1123).

As used herein the term “chimeric” means that the antibody or antigen-binding portion includes sequences from two different species.

In one embodiment, the domain antibody comprises a human framework regions and at least one New World primate CDRs, more preferably marmoset CDRs.

Preferably, the New World primate is selected from the group consisting of marmosets, tamarins, squirrel monkey, titi monkey, spider monkey, woolly monkey, capuchin, uakaris, sakis, night or owl monkey and the howler monkey. More preferably, the New World primate is a marmoset.

Methods of producing chimeric antibodies according to the invention will be familiar to persons skilled in the art, see for example, U.S. Pat. No. 4,816,567, U.S. Pat. No. 5,585,089 and U.S. Ser. No. 20030039649 which are incorporated herein by reference in their entirety. Such methods require the use of standard recombinant techniques.

It is preferred that the antibody or antigen-binding portion thereof according to the present invention has predicted low immunogenicity in a human host.

By “low immunogenicity” it is meant that the antibody does not raise an antibody response in at least the majority of individuals receiving the antibody of sufficient magnitude to reduce the effectiveness of continued administration of the antibody for a sufficient time to achieve therapeutic efficacy.

The level of immunogenicity in humans may predicted using the MHC class II binding prediction program Propred (http://www.imtech.res.in/raghava/propred) using a 1% threshold value analysis of all alleles. Other programs which may be used include:

Rankpep (http://bio.dfci.harvard.edu/Tools/rankpep.html)

Epibase (Algonomics proprietary software: algonomics.com)

Low immunogenicity molecules will contain no or low numbers of peptides predicted to bind to MHC class II alleles that are highly expressed in the target population (Flower D R, Doytchinova I A. 2004) Immunoinformatics and the prediction of immunogenicity, Drug Discov Today, 9(2): 82-90).

Reduced immunogenicity molecules will contain no or a reduced numbers of peptides predicted to bind to MHC class II alleles that are highly expressed in the target population, relative to the starting donor molecule.

Functional analysis of MHC class II binding can be performed by generating overlapping peptides corresponding to the protein of interest and testing these for their ability to evoke T cell activation (T cell proliferation assay) or displace a reporter peptide, a known MHC class II-binding peptide (Hammer J et al., 1994, J. Exp. Med., 180:2353).

The present invention is further based on a method for amplification of New World primate immunoglobulin genes, for example by polymerase chain reaction (PCR) from nucleic acid extracted from New World primate lymphocytes using primers specific for heavy and light chain variable region gene families. The amplified variable region is then cloned into an expression vector containing a human or primate constant region gene for the production of New World primate chimeric recombinant antibody. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning: a laboratory manual, second edition, Cold Spring Harbor, N.Y. (1989).

Suitable expression vectors will be familiar to those skilled in the art. The New World primate lymphocytes producing the immunoglobulins are typically immortalised by fusion with a myeloma cell line to generate a hybridoma.

Preferred mammalian host cells for expressing the recombinant antibodies of the invention include Chinese Hamster Ovary (CHO), NS0 myeloma cells, COS cells and SP2 cells.

In addition to mammalian expression systems, the present invention also contemplates the use of non-mammalian expression systems such as those which are plant or prokaryotic (bacterial) derived. Such expression systems would be familiar to persons skilled in the art.

The repertoire of V_{H}, V_{L} and constant region domains can be a naturally occurring repertoire of immunoglobulin sequences or a synthetic repertoire. A naturally occurring repertoire is one prepared, for example, from immunoglobulin expressing cells harvested from one or more primates. Such repertoires can be naïve ie. prepared from newborn immunoglobulin expressing cells, or rearranged ie. prepared from, for example, adult primate B cells. If desired, clones identified from a natural repertoire, or any repertoire that bind the target antigen are then subject to mutagenesis and further screening in order to produce and select variants with improved binding characteristics.

Synthetic repertoires of immunoglobulin variable domains are prepared by artificially introducing diversity into a cloned variable domain. Such affinity maturation techniques will be familiar to persons skilled in the art (Irving R. A. et al. (2001) Ribosome display and affinity maturation: from antibodies to single V-domains and steps towards cancer therapeutics, Journal of Immunological Methods, 248: 31-45).

The variable region, or a CDR thereof, of a New World primate antibody gene may be cloned by providing nucleic acid eg. cDNA, providing a primer complementary to the cDNA sequence encoding a 5′ leader sequence of an antibody gene, contacting that cDNA and the primer to form a hybrid complex and amplifying the cDNA to produce nucleic acid encoding the variable region (or CDR region) of the New World primate antibody gene.

It will be appreciated by persons skilled in the art of the present invention, the non-New World primate variable region sequence may be used as an acceptor for grafting New World primate sequences, in particular, CDR sequences using standard recombinant techniques. For example, U.S. Pat. No. 5,585,089 describes methods for creating low immunogenicity chimeric antibodies that retain the high affinity of the non-human parent antibody and contain one or more CDRs from a donor immunoglobulin and a framework region from a human immunoglobulin. United States publication no. 20030039649 describes a humanisation method for creating low immunogenicity chimeric antibodies containing CDR sequences from a non-human antibody and framework sequences of human antibodies based on using canonical CDR structure types of the non-human antibody in comparison to germline canonical CDR structure types of human antibodies as the basis for selecting the appropriate human framework sequences for a humanised antibody. Accordingly, these principles can be applied to the grafting of one or more New World primate CDRs into a non-New World primate acceptor variable region.

The CDR sequences may be obtained from the genomic DNA isolated from an antibody, or from sequences present in a database e.g. The National Centre for Biotechnology Information protein and nucleotide databases, The Kabat Database of Sequences of Proteins of Immunological Interest. The CDR sequence may be a genomic DNA or a cDNA.

Methods for grafting a replacement CDR(s) into an acceptor variable sequence will be familiar to persons skilled in the art of the present invention. Typically, the CDRs will be grafted into acceptor variable region sequences for each of a variable light chain and a variable heavy chain or a single chain in the case of a domain antibody. The preferred method of the present invention involves replacement of either CDR1 or, more preferably, CDR2 in a variable region sequence via primer directed mutagenesis. The method consists of annealing a synthetic oligonucleotide encoding a desired mutation to a target region where it serves as a primer for initiation of DNA synthesis in vitro, extending the oligonucleotide by a DNA polymerase to generate a double-stranded DNA that carries the desired mutation, and ligating and cloning the sequence into an appropriate expression vector (Sambrook, Joseph; and David W. Russell (2001). Molecular Cloning: A Laboratory Manual, 3rd ed., Cold Spring Harbor, N.Y.: Cold Spring Harbor Laboratory Press).

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein as is known to the skilled artisan.

The constant region sequence (Fc portion) is preferably obtained from a human or non-human primate immunoglobulin sequence. The primate sequence may be a New World primate or an Old World primate sequence. Suitable Old World primates include chimpanzee, or other hominid ape eg. gorilla or orang utan, which because of their close phylogenetic proximity to humans, share a high degree of homology with the human constant region sequence. Sequences which encode for human or primate constant regions are available from databases including e.g. The National Centre for Biotechnology Information protein and nucleotide databases, The Kabat Database of Sequences of Proteins of Immunological Interest.

The antibody or antigen-binding portion according to the invention is capable of binding to a human or non-human antigen.

Preferably, the antigen to which the chimeric antibody or antigen-binding portion thereof binds, is peptide, protein, carbohydrate, glycoprotein, lipid or glycolipid in nature, selected from a tumour-associated antigen including carcinoembryonic antigen, EpCAM, Lewis-Y, Lewis-Y/b, PMSA, CD20, CD30, CD33, CD38, CD52, CD154, EGF-R, Her-2, TRAIL and VEGF receptors, an antigen involved in an immune or inflammatory disease or disorder including CD3, CD4, CD25, CD40, CD49d, MHC class I, MHC class II, GM-CSF, interferon-γ, IL-1, IL-12, IL-13, IL-23, TNF-α, and IgE, an antigen expressed on a host cell including glycoprotein IIb/IIIa, P-glycoprotein, purinergic receptors and adhesion receptors including CD11a, CD11b, CD11c, CD18, CD56, CD58, CD62 or CD 144, an antigen comprising a cytokine, chemokine, growth factor or other soluble physiological modulator or a receptor thereof including eotaxin, IL-6, IL-8, TGF-β, C3a, C5a, VEGF, NGF and their receptors, an antigen involved in central nervous system diseases or disorders including β-amyloid and prions, an antigen of non-human origin such as microbial, nanobial or viral antigens or toxins including respiratory syncitial virus protein F, anthrax toxin, rattle snake venom and digoxin; wherein the chimeric antibody acts as an agonist or antagonist or is active to either deplete (kill or eliminate) undesired cells (eg. anti-CD4) by acting with complement, or killer cells (eg. NK cells) or is active as a cytotoxic agent or to cause Fc-receptor binding by a phagocyte or neutralizes biological activity of its target.

More preferably, the antigen is TNFα, preferably human TNFα.

Alternatively the chimeric antibody or antigen-binding portion thereof may bind a non-human antigen. Preferrably the non-human antigen is selected from the group consisting of respiratory syncytial virus F protein, cytomegalovirus, snake venoms and digoxin.

The term “binds to” as used herein, is intended to refer to the binding of an antigen by an immunoglobulin variable region of an antibody with a dissociation constant (K_{D}) of 1 μM or lower as measured by surface plasmon resonance analysis using, for example a BIAcore™ surface plasmon resonance system and BIAcore™ kinetic evaluation software (eg. version 2.1). The affinity or dissociation constant (K_{D}) for a specific binding interaction is preferably about 500 nM to about 50 pM, more preferably about 500 nM or lower, more preferably about 300 nM or lower and preferably at least about 300 nM to about 50 pM, about 200 nM to about 50 pM, and more preferably at least about 100 nM to about 50 pM, about 75 nM to about 50 pM, about 10 nM to about 50 pM.

The antibodies of the present invention are advantageous in human therapy because the likelihood of induction of a human anti-antibody response will be reduced.

Recombinant antibodies produced according to the invention that bind a target antigen can be identified and isolated by screening a combinatorial immunoglobulin library (eg a phage display library) to isolate library members that exhibit the desired binding specificity and functional behaviour. It will be understood that all approaches where antigen-binding portions or derivatives of antibodies are used, eg Fabs, scFv and V domains or domain antibodies, lie within the scope of the present invention. The phage display technique has been described extensively in the art and examples of methods and compounds for generating and screening such libraries and affinity maturing the products of them can be found in, for example, Barbas et al. (1991) PNAS 88:7978-7982; Clarkson et al. (1991) Nature 352:624:628; Dower et al. PCT. 91/17271, U.S. Pat. No. 5,427,908, U.S. Pat. No. 5,580,717 and EP 527,839; Fuchs et al. (1991) Bio/Technology 9:1370-1372; Garrad et al. (1991) Bio/Technology 9:1373:1377; Garrard et al. PCT WO 92/09690; Gram et al. (1992) PNAS 89:3576-3580; Griffiths et al. (1993) EMBO J 12:725:734; Griffiths et al. U.S. Pat. No. 5,885,793 and EP 589,877; Hawkins et al. (1992) J Mol Biol 226:889-896; Hay et al. (1992) Hum Antibod Hybridomas 3:81-85; Hoogenboom et al. (1991) Nuc Acid Res 19:4133-4137; Huse et al. (1989) Science 246:1275-1281; Knappik et al. (2000) J Mol Biol 296:57-86; Knappik et al. PCT WO 97/08320; Ladner et al. U.S. Pat. No. 5,223,409, No. 5,403,484, No. 5,571,698, No. 5,837,500 and EP 436,597; McCafferty et al. (1990) Nature 348:552-554; McCafferty et al. PCT. WO 92/01047, U.S. Pat. No. 5,969,108 and EP 589,877; Salfeld et al. PCT WO 97/29131, U.S. Provisional Application No. 60/126,603; and Winter et al. PCT WO 92/20791 and EP 368,684;

Recombinant libraries expressing the antibodies of the invention can be expressed on the surface of microorganisms eg. yeast or bacteria (see PCT publications WO99/36569 and 98/49286).

The Selected Lymphocyte Antibody Method or SLAM as it is referred to in the state of the art, is another means of generating high affinity antibodies rapidly. Unlike phage display approaches all antibodies are fully divalent. In order to generate New World primate antibodies, New World primates are immunised with a human antigen eg. a TNFα polypeptide. Following immunisation cells are removed and selectively proliferated in individual micro wells. Supernatants are removed from wells and tested for both binding and function. Gene sequences can be recovered for subsequent manipulations eg. humanisation, Fab fragment, scFv or domain antibody generation. Thus another example is the derivation of the ligand of the invention by SLAM and its derivatives (Babcook, J. S. et al 1996, Proc. Natl. Acad. Sci, USA 93; 7843-7848, U.S. Pat. No. 5,627,052 and PCT publication WO92/02551). Adaptations of SLAM, such as the use of alternatives to testing supernatants such as panning, also lie within the scope of this invention.

In one expression system the recombinant peptide/protein library is displayed on ribosomes (for examples see Roberts, R W and Szostak, J. W.1997. Proc. Natl. Acad. Sci. USA. 94:12297-123202 and PCT Publication No. WO98/31700). Thus another example involves the generation and in vitro transcription of a DNA library (eg of antibodies and derivatives) preferably prepared from immunised cells, but not so limited), translation of the library such that the protein and “immunised” mRNAs stay on the ribosome, affinity selection (eg by binding to RSP), mRNA isolation, reverse translation and subsequent amplification (eg by polymerase chain reaction or related technology). Additional rounds of selection and amplification can be coupled as necessary to affinity maturation through introduction of somatic mutation in this system or by other methods of affinity maturation as known in the state of the art.

Another example sees the application of emulsion compartmentalisation technology to the generation of the antibodies of the invention. In emulsion compartmentalisation, in vitro and optical sorting methods are combined with co-compartmentalisation of translated protein and its nucleotide coding sequence in aqueous phase within an oil droplet in an emulsion (see PCT publications no's WO99026711 and WO0040712). The main elements for the generation and selection of antibodies are essentially similar to the in vitro method of ribosome display.

The antibody or antigen-binding portion thereof according to the invention can be derivatised or linked to another functional molecule. For example, the antibody or antigen-binding portion can be functionally linked by chemical coupling, genetic fusion, noncovalent association or otherwise, to one or more other molecular entities, such as another antibody, a detectable agent, a cytotoxic agent, a pharmaceutical agent, and/or a protein or peptide that can mediate association of the antibody or antigen-binding portion thereof with another molecule (such as a streptavidin core region or a polyhistidine tag).

Useful detectable agents with which an antibody or antigen-binding portion thereof may be derivatised include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatised with detectable enzymes such as alkaline phosphatase, horseradish peroxidase, glucose oxidase and the like. When an antibody is derivatized with a detectable enzyme, it is detected by adding additional reagents that the enzyme uses to produce a detectable reaction product. An antibody may also be derivatised with biotin, and detected through indirect measurement of avidin or streptavidin binding.

The present invention also extends to PEGylated antibodies or antibody-binding portion which provide increased half-life and resistance to degradation without a loss in activity (eg. binding affinity) relative to non-PEGylated antibody polypeptides.

The antibody or antibody-binding portion as described herein can be coupled, using methods known in the art, to polymer molecules (preferably PEG) useful for achieving the increased half-life and degradation resistance properties. Polymer moieties which can be utilised in the invention can be synthetic or naturally occurring and include, but are not limited to, straight or branched chain polyalkylene, polyalkenylene or polyoxyalkylene polymers, or a branched or unbranched polysaccharide such as a homo-or heteropolysaccharide. Preferred examples of synthetic polymers which can be used in the invention include straight or branched chain poly(ethylene glycol) (PEG), poly(propylene glycol), or poly(vinyl alcohol) and derivatives or substituted forms thereof. Particularly preferred substituted polymers for linkage to antibodies as described herein include substituted PEG, including methoxy(polyethylene glycol). Naturally occurring polymer moieties which can be used in addition to or in place of PEG include lactose, amylose, dextran, or glycogen, as well as derivatives thereof which would be recognised by persons skilled in the art.

Derivatized forms of polymer molecules include, for example, derivatives which have additional moieties or reactive groups present therein to permit interaction with amino acid residues of the antibody polypeptides described herein. Such derivatives include N-hydroxylsuccinimide (NHS) active esters, succinimidyl propionate polymers, and sulfhy selective reactive agents such as maleimide, vinyl sulfone, and thiol. Particularly preferred derivatized polymers include, but are not limited to PEG polymers having the formulae: PEG-O—CH₂CH₂CH₂—CO₂-NHS; PEG-O—CH₂-NHS; PEG-O—CH₂CH₂—CO₂-NHS; PEG—S—CH₂CH₂—CO-NHS; PEG-O₂CNH—CH(R)—CO₂-NHS; PEG-NHCO-CH₂CH₂—CO-NHS; and PEG-O—CH₂—CO₂-NHS; where R is (CH₂)₄)NHCO₂(mPEG). PEG polymers can be linear molecules, or can be branched wherein multiple PEG moieties are present in a single polymer.

The reactive group (e.g., MAL, NHS, SPA, VS, or Thiol) may be attached directly to the PEG polymer or may be attached to PEG via a linker molecule.

The size of polymers useful in the invention can be in the range of between 500 Da to 60 kDa, for example, between 1000 Da and 60 kDa, 10 kDa and 60 kDa, 20 kDa and 60 kDa, 30 kDa and 60 kDa, 40 kDa and 60 kDa, and up to between 50 kDa and 60 kDa. The polymers used in the invention, particularly PEG, can be straight chain polymers or may possess a branched conformation.

The polymer (PEG) molecules useful in the invention can be attached to an antibody or antigen-binding portion thereof using methods which are well known in the art. The first step in the attachment of PEG or other polymer moieties to an antibody polypeptide monomer or multimer of the invention is the substitution of the hydroxyl end-groups of the PEG polymer by electrophile-containing functional groups. Particularly, PEG polymers are attached to either cysteine or lysine residues present in the antibody polypeptide monomers or multimers. The cysteine and lysine residues can be naturally occurring, or can be engineered into the antibody polypeptide molecule. For example, cysteine residues can be recombinantly engineered at the C-terminus of an antibody polypeptide, or residues at specific solvent accessible locations in an antibody polypeptide can be substituted with cysteine or lysine.

The antibody may be linked to one or more molecules which can increase its half-life in vivo. These molecules are linked to the antibody at a site on the antibody other than the antigen binding site, so that they do not interfere/sterically hinder the antigen-binding site. Typically, such molecules are polypeptides which occur naturally in vivo and which resist degradation or removal by endogenous mechanisms. It will be obvious to one skilled in the art that fragments or derivatives of such naturally occurring molecules may be used, and that some may not be polypeptides. Molecules which increase half life may be selected from the following:

(a) proteins from the extracellular matrix, eg. collagen, laminin, integrin and fibronectin;

(b) proteins found in blood, eg. fibrin α-2 macroglobulin, serum albumin, fibrinogen A, fibrinogen B, serum amyloid protein A, heptaglobin, protein, ubiquitin, uteroglobulin, β-2 microglobulin, plasminogen, lysozyme, cystatin C, alpha-1-antitrypsin and pancreatic kypsin inhibitor;

(c) immune serum proteins, eg. IgE, IgG, IgM;

(d) transport proteins, eg. retinol binding protein, α-1 microglobulin;

(e) defensins, eg. beta-defensin 1, Neutrophil defensins 1, 2 and 3;

(f) proteins found at the blood brain barrier or in neural tissues, eg. melanocortin receptor, myelin, ascorbate transporter;

(g) transferrin receptor specific ligand-neuropharmaceutical agent fusion proteins (see U.S. Pat. No. 5,977,307); brain capillary endothelial cell receptor, transferrin, transferrin receptor, insulin, insulin-like growth factor 1 (IGF 1) receptor, insulin-like growth factor 2 (IGF 2) receptor, insulin receptor;

(h) proteins localised to the kidney, eg. polycystin, type IV collagen, organic anion transporter K1, Heymann's antigen;

(i) proteins localised to the liver, eg. alcohol dehydrogenase, G250;

(j) blood coagulation factor X;

(k) α-1 antitrypsin;

(l) HNF 1α;

(m) proteins localised to the lung, eg. secretory component (binds IgA);

(n) proteins localised to the Heart, eg. HSP 27;

(o) proteins localised to the skin, eg, keratin;

(p) bone specific proteins, such as bone morphogenic proteins (BMPs) eg. BMP-2, -4, -5, -6, -7 (also referred to as osteogenic protein (OP-1) and -8 (OP-2);

(q) tumour specific proteins, eg. human trophoblast antigen, herceptin receptor, oestrogen receptor, cathepsins eg cathepsin B (found in liver and spleen);

(r) disease-specific proteins, eg. antigens expressed only on activated T-cells: including LAG-3 (lymphocyte activation gene); osteoprotegerin ligand (OPGL) see Nature 402, 304-309, 1999; OX40 (a member of the TNFα receptor family, expressed on activated T cells and the only costimulatory T cell molecule known to be specifically up-regulated in human T cell leukaemia virus type-I (HTLV-I)-producing cells—see J. Immunol. 2000 Jul 1;16561):263-70; metalloproteases (associated with arthritis/cancers), including CG6512 Drosophila, human paraplegin, human FtsH, human AFG3L2, murine ftsH; angiogenic growth factors, including acidic fibroblast growth factor (FGF-1), basic fibroblast growth factor (FGF-2), Vascular endothelial growth factor/vascular permeability factor (VEGF/VPF), transforming growth factor-α (TGF-α), tumor necrosis factor-alpha (TNFα), angiogenin, interleukin-3 (IL-3), interleukin-8 (IL-8), platelet derived endothelial growth factor (PD-ECGF), placental growth factor (P1GF), midkine platelet-derived growth factor-BB (PDGF), fractalkine;

(s) stress proteins (heat shock proteins);

(t) proteins involved in Fc transport; and

(u) vitamins eg B12, Biotin.

In another aspect, the invention provides a pharmaceutical composition comprising an effective amount of the chimeric antibody or antigen-binding portion thereof according to the present invention, together with a pharmaceutically acceptable excipient or diluent.

A “pharmaceutically acceptable excipient or diluent” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, and the like as well as combinations thereof. In many cases it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers.

The term “effective amount” refers to an amount of an antibody or antigen binding portion thereof (including pharmaceutical compositions comprising the antibody or antigen binding portion thereof) sufficient to treat a specified disease or disorder or one or more of its symptoms and/or to prevent the occurrence of the disease or disorder.

The term “diagnostically effective amount” or “amounts effective for diagnosis” and cognates thereof, refers to an amount of a antibody or antigen binding portion thereof (including pharmaceutical compositions comprising the antibody or antigen binding portion thereof) sufficient to diagnose a specified disease or disorder and/or one or more of its manifestations, where diagnosis includes identification of the existence of the disease or disorder and/or detection of the extent or severity of the disease or disorder. Often, diagnosis will be carried out with reference to a baseline or background detection level observed for individuals without the disease or disorder. Levels of detection above background or baseline levels (elevated levels of detection) are indicative of the presence and, in some cases, the severity of the condition.

When used with respect to methods of treatment and the use of the antibody or antigen binding portion thereof (including pharmaceutical compositions comprising the antibody or antigen binding portion thereof), an individual “in need thereof” may be an individual who has been diagnosed with or previously treated for the disease or disorder to be treated. With respect to methods of diagnosis, an individual “in need thereof” may be an individual who is suspected to have a disease or disorder, is at risk for a disease or disorder, or has previously been diagnosed with the disease or disorder (e.g., diagnosis can include monitoring of the severity (e.g., progression/regression) of the disease or disorder over time and/or in conjunction with therapy).

It is preferred that the chimeric antibody or antigen-binding portion thereof blocks or stimulates receptors functions or neutralizes active soluble products, such as one or more of the interleukins, TNFα or C5a. More preferably, the active soluble product is human TNFα.

The composition may be in a variety of forms, including liquid, semi-solid or solid dosage forms, such as liquid solutions (eg injectable and infusible solutions), dispersions or suspensions, tablets, pills, powders, liposomes or suppositories. Preferably, the composition is in the form of an injectable solution for immunization. The administration may be intravenous, subcutaneous, intraperitoneal, intramuscular, transdermal, intrathecal, and intra-arterial. Preferably the dosage form is in the range of from about 0.001 mg to about 10 mg/kg body weight administered daily, weekly, bi- or tri-weekly or monthly, more preferably about 0.05 to about 5 mg/kg body weight weekly.

The composition may also be formulated as a sterile powder for the preparation of sterile injectable solutions.

In certain embodiments, the active compound may be prepared with a carrier that will protect the compound against rapid release, such as a controlled release formulation, including implants, transdermal patches, and microencapsulated delivery systems. Compatible polymers may be used such as ethylene vinyl acetate, polyanhydrides, polyglycolic acid, collagen, polyorthoesters or polylactic acid.

The composition may also be formulated for oral administration. In this embodiment, the antibody may be enclosed in a hard or soft shell gelatin capsule, compressed into tablets, or incorporated directly into the subject's diet.

The composition may also be formulated for rectal administration.

The antibody may be administered in order to bind to and identify selected cells in vitro and in vivo, to bind to and destroy selected cells in vivo, or in order to penetrate into and destroy selected cells in vivo. Alternatively, the antibody may be used as an immunotoxin to deliver a cytotoxic agent eg. a toxin or chemotherapeutic agent to a particular cell type such as a tumour cell. Production of immunotoxins would be familiar to persons skilled in the art.

Cytotoxic agents commonly used to generate immunotoxins include radioactive isotopes such as ¹¹¹In or ⁹⁰Y, selenium, ribonucleases, binding domain—deleted truncated microbial toxins such as Pseudomonas exotoxin or Diphtheria toxin, tubulin inhibitors such as calicheamicin (ozagamicin), maytansinoids (including DM-1), auristatins, and taxoids, ribosome inactivating proteins such as ricin, ebulin I, saporin and gelonin, and prodrugs such as melphalan.

In the preferred embodiment, the composition is administered to a human.

The present invention also provides for the use of the chimeric antibody or antigen-binding portion thereof in a diagnostic application for detecting an antigen associated with a particular disease or disorder.

More particularly, the invention provides for the use of the chimeric antibody or antigen-binding portion thereof in a method for diagnosing a subject having an antigen associated with a particular disease or disorder, comprising administering to said subject a diagnostically effective amount of a pharmaceutical composition according to the third aspect. Preferably the subject is a human.

For example, the chimeric antibody or antigen-binding fragment thereof, preferably labelled, can be used to detect the presence of an antigen, or elevated levels of an antigen (e.g. TNFα) in a biological sample, such as serum or plasma using a convention immunoassay, such as an enzyme linked immunosorbent assay (ELISA), a radioimmunoassay (RIA) or tissue immunohistochemistry.

Preferably, the antigen to which the chimeric antibody or antigen-binding portion thereof binds, is peptide, protein, carbohydrate, glycoprotein, lipid or glycolipid in nature, selected from a tumour-associated antigen including carcinoembryonic antigen, EpCAM, Lewis-Y, Lewis-Y/b, PMSA, CD20, CD30, CD33, CD38, CD52, CD154, EGF-R, Her-2, TRAIL and VEGF receptors, an antigen involved in an immune or inflammatory disease or disorder including CD3, CD4, CD25, CD40, CD49d, MHC class I, MHC class II, GM-CSF, interferon-γ, IL-1, IL-12, IL-13, IL-23, TNF-α, and IgE, an antigen expressed on a host cell including glycoprotein IIb/IIIa, P-glycoprotein, purinergic receptors and adhesion receptors including CD11a, CD11b, CD11c, CD18, CD56, CD58, CD62 or CD 144, an antigen comprising a cytokine, chemokine, growth factor or other soluble physiological modulator or a receptor thereof including eotaxin, IL-6, IL-8, TGF-β, C3a, C5a, VEGF, NGF and their receptors, an antigen involved in central nervous system diseases or disorders including β-amyloid and prions, an antigen of non-human origin such as microbial, nanobial or viral antigens or toxins including respiratory syncitial virus protein F, anthrax toxin, rattle snake venom and digoxin; wherein the chimeric antibody acts as an agonist or antagonist or is active to either deplete (kill or eliminate) undesired cells (eg. anti-CD4) by acting with complement, or killer cells (eg. NK cells) or is active as a cytotoxic agent or to cause Fc-receptor binding by a phagocyte or neutralizes biological activity of its target.

The anti-human TNFα chimeric antibody or antigen binding portion thereof according to the invention may also be used in cell culture applications where it is desired to inhibit TNFα activity.

The present invention also provides a method for treating a disease or disorder characterised by human TNFα activity in a human subject, comprising administering to the subject in need thereof a pharmaceutical composition according to the present invention in which the chimeric antibody or antigen-binding portion thereof binds TNFα.

The term “disease or disorder characterised by human TNFα activity” as used herein is intended to include diseases or disorders in which the presence of TNFα in a subject suffering from the disease or disorder has been shown to be or is suspected of being either responsible for the pathophysiology of the disease or disorder or a factor that contributes to the worsening of the disease or disorder. Accordingly, a disease or disorder in which TNFα activity is detrimental is a disease or disorder in which inhibition of TNFα activity is expected to alleviate symptoms and/or progression of the disease or disorder. Such diseases or disorders may be evidenced, for example, by an increase in the concentration of TNFα in a biological fluid of a subject suffering from the disease or disorder (e.g., an increase in the concentration of TNFα in serum, plasma, synovial fluid etc of the subject), which can be detected, for example, using a chimeric antibody of the invention specific for TNFα.

A disease or disorder characterised by human TNFα activity is intended to include diseases or disorders and other disease or disorder in which the presence of TNFα in a subject suffering from the disease or disorder has been shown to be, or is suspected of being, either responsible for the pathophysiology of the disease or disorder or a factor which contributes to a worsening of the disease or disorder. Preferably, the disease or disorder characterised by human TNFα activity is selected from the group consisting of sepsis, including septic shock, endotoxic shock, gram negative sepsis and toxic shock syndrome; autoimmune disease, including rheumatoid arthritis, rheumatoid spondylitis, osteoarthritis, psoriasis and gouty arthritis, allergy, multiple sclerosis, autoimmune diabetes, autoimmune uveitis and nephrotic syndrome; infectious disease, including fever and myalgias due to infection and cachexia secondary to infection; graft versus host disease; tumour growth or metastasis; pulmonary disease including adult respiratory distress syndrome, shock lung, chronic pulmonary inflammatory disease, pulmonary sarcoidosis, pulmonary fibrosis and silicosis; inflammatory bowel disease including Crohn's disease and ulcerative colitis; cardiac disease; inflammatory bone disease, hepatitis, coagulation disturbances, burns, reperfusion injury, keloid formation and scar tissue formation.

Supplementary active compounds can also be incorporated into the composition. The antibody or antibody-binding fragment may be co-formulated with and/or administered simultaneously, separately or sequentially with one or more additional therapeutic agents eg. antibodies that bind to other targets such as cytokines or cell surface molecules or alternatively one or more chemical agents that inhibit human TNFα production or activity.

In another aspect, the invention provides a kit comprising a therapeutically effective amount of a chimeric antibody or antigen-binding portion of the invention, or a pharmaceutical composition comprising a therapeutically effective amount of a chimeric antibody or antigen-binding portion thereof, together with packaging and instructions for use. In certain embodiments, the instructions for use include instructions for how to effectively administer a therapeutic amount of the chimeric antibody or antigen-binding portion of the invention.

Throughout this specification the word “comprise”, or variations such as “comprises” or comprising, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

In order that the nature of the present invention may be more clearly understood, preferred forms thereof will now be described with reference to the following non-limiting examples.

### EXAMPLE 1

### Fusion of a Marmoset Variable Region to a Human Constant Region

### Materials and Methods

### Gene Synthesis and Cloning

The V_{H} chain (Accession Number: AAM54057, SEQ ID NO: 1) of the MOG specific marmoset derived antibody was expressed with a human constant region (human IgG1 heavy chain C_{H}1, hinge, C_{H}2 & C_{H}3 domains (such as NCBI accession number P01857) (SEQ ID NO: 2)). This was achieved by back translation of the amino acid sequence into a DNA sequence which was optimized for mammalian cell expression using GeneOptimizer technology and synthesized de novo by assembly of synthetic oligonucleotides (GeneArt, Germany). During DNA sequence optimisation the specific restriction enzyme sites Asc I and Tth 111I were included to allow for future manipulation of the V_{H} region. Following gene synthesis the whole sequence including a Kozak sequence was cloned into the multiple cloning site of the pEE6.4 GS accessory vector (Lonza Biologics). The V_{L} chain (Accession Number: AAM54058, SEQ ID NO: 3) of the MOG specific marmoset derived antibody was expressed with a human kappa light chain constant region (such as NCBI accession number AAA58989) (SEQ ID NO: 4). DNA encoding the light chain (V_{L}-Kappa) amino acid sequence was prepared as described above for the heavy chain. During DNA sequence optimization and synthesis the specific restriction enzyme sites Bsi WI/Rsr II were included to allow future manipulation of the V_{L} region. Following gene synthesis the whole sequence including a Kozak sequence was cloned into the multiple cloning site of the pEE12.4 GS expression vector (Lonza Biologics). For stable expression the two single gene vectors (pEE6.4-V_{H}-IgG₁ and pEE12.4-V_{L}-Kappa) were combined into a double gene vector. This was done by digesting out of the pEE6.4 backbone the heavy chain expression cassette (hCMV-MIE promoter, Kozak sequence, marmoset V_{H}, human constant region and SV40 polyA site) using Not I and BamH I. The resultant fragment was subcloned using Not I and BamH I sites into the pEE12.4-V_{L}-Kappa vector downstream of the light chain expression cassette (hCMV-MIE promoter, Kozak sequence, marmoset V_{L}, human Kappa constant region and SV40 polyA site) creating a vector expressing both the heavy and light chains of AB138 (SEQ ID NOs: 5 and 6).

### Transfection

For each transfection 175 μl of Lipofectamine 2000 was added to 5 mL of Optimem I media (Invitrogen Cat Nos. 11668-027 and 31985-062) in a well of a 6 well plate. In a second well 70 μl of the expression vector (70 μg) was added to 5 mL of Optimem I media. Following a 5 minute room temperature incubation, the contents of the two wells were mixed together and left for a further 20 minute incubation. Following this second incubation the whole transfection mixture was added to a T175 tissue culture flask containing the CHOK1SV cells. Cells were incubated for 72 to 96 hours and supernatants harvested. Supernatants were centrifuged at 4,000×g for 5 minutes to pellet cell debris, and were filter sterilised through 0.22 μm cartridge filter.

### Antibody Purification

The supernatant was passed over a HiTrap Protein A column (Amersham Biosciences, Cat No: 17-0402-01) three times at a flow rate of 1 mL/min. The column was then washed with 20 mM sodium phosphate for 40 mins at 1 mL/min. The antibody was eluted with 0.1 M citric acid pH 3.5 with fractions collected and immediately neutralised with 1M Tris-HCl pH 9.0. Antibody samples were then desalted on a PD-10 column (Amersham Biosciences, Cat No: 17-0851-01). Analysis of the antibody by SDS-PAGE and size-exclusion HPLC confirmed the correct molecular weight, presence of assembled antibody and the concentration of antibody.

### Western Blot Analysis

The ability of AB138 to retain binding to the antigen of M26, rat MOG (myelin-oligodendrocyte glycoprotein), was investigated by Western Blot. 130 mg of rat spinal cord (IMVS, Australia) was homogenized in 1.8 ml CelLytic M Cell Lysis Reagent (SIGMA, C2978) and incubated for 30 minutes at 4° C. Further homogenization was performed by drawing the lysate through a 27 g½ needle several times followed by centrifugation at 4° C. and 13000 g for 30 minutes. The pellet and supernatant was diluted into SDS-PAGE sample buffer (125 mM Tris-HCl pH 6.8, 5% SDS, 0.25% bromophenol blue, 25% glycerol). Along with this 200 μl CHOK1SV cells at 1×10⁶ viable cells per ml were spun down at 13000×g at 4° C. for 1 minute and resuspended in 200 μl CelLytic M Cell Lysis Reagent (SIGMA). Following centrifugation at 4° C. and 13000×g for 30 minutes the supernatant was mixed with the appropriate amount of SDS-PAGE sample buffer. All samples, along with a sample of molecular weight markers, were run on a 4-20% Novex pre-cast gel (Invitrogen, Australia) for 2 hours at 120V. Proteins were then transferred to PVDF (BioRad, Australia) using a western blot apparatus in 1×Tris-Glycine Buffer with 20% methanol (BioRad, Cat 161+−0771) at 4° C. at 250 mA for 2 hours. The membrane was then blocked by incubation with 5% skim milk powder in PBS for 1 h at room temperature. The membrane was then washed with 1×PBS three times followed by an overnight incubation at 4° C. with AB138 in PBS at 10 ug/mL. After washing, the membrane was incubated with Goat Anti-human IgG (H+L) HRP conjugate (Sigma, Australia) diluted 1:5000 in 1×PBS for 1 hour at room temperature. Following washing, bound antibody was detected using the ECL Western Blotting Analysis System, (Amersham Biosciences Cat: RPN2109). A parallel experiment was performed in which AB138 was replaced with an isotype-matched irrelevant specificity negative control antibody (anti-TNFα monoclonal antibody) in order to identify any non-specific binding events.

### Results

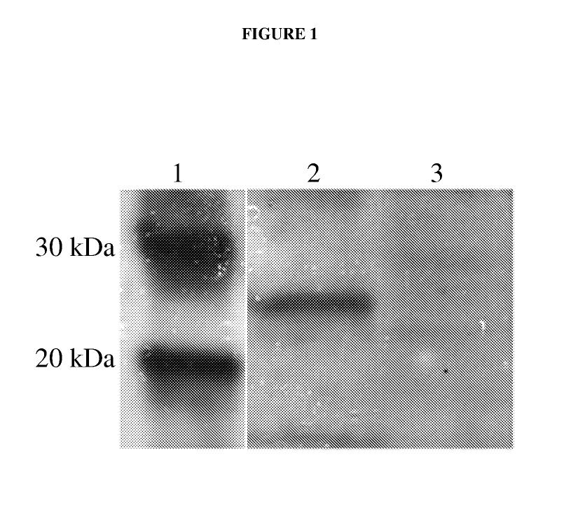

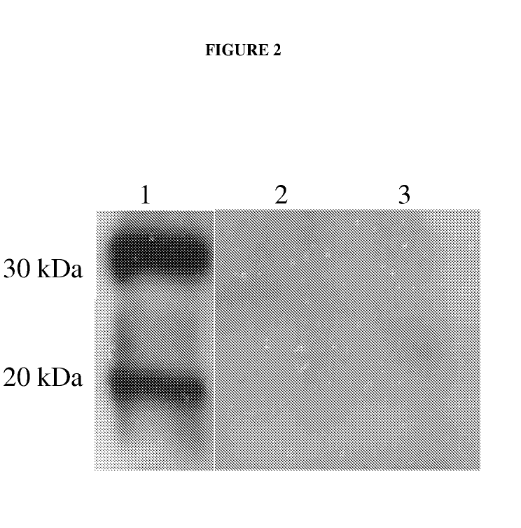

After successful protein expression and purification, western blot analysis was performed on AB138 to determine if it retained binding affinity to rat MOG. AB138 bound a protein with approximate size of 25 kDa present in the rat spinal cord cleared lysate, a protein not present in cleared CHOK1SV lysate (FIG. 1). The negative control antibody did not bind to protein present in either lysate indicating the interaction between AB138 and the protein of size 25 kDa was not due to artifact or non-specific binding events associated with the human constant region (FIG. 2). This protein matches the expected size of rat MOG minus the signal sequence (24.9 kDa). This result indicates that AB138 retained affinity for rat MOG present in rat spinal cord lysate and demonstrates that a marmoset human fusion antibody can retain antigen binding ability.

It can be appreciated by someone skilled in the art that rat MOG could be produced using recombinant DNA technology and the ability of AB138 to bind rat MOG determined in binding assays such as ELISA or Biacore analysis.

### EXAMPLE 2

### CDR2 Substitution of a Domain Antibody

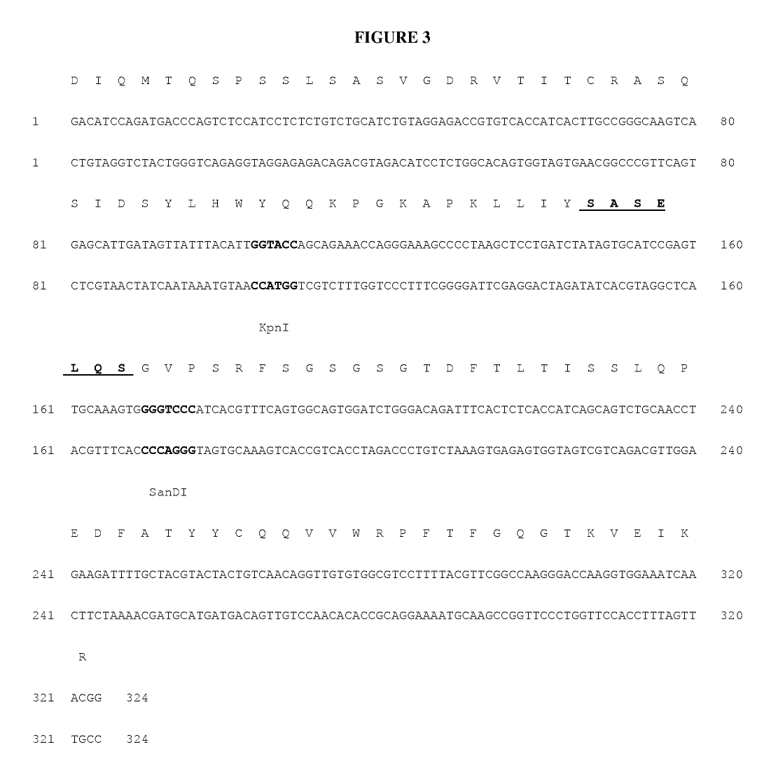

Standard recombinant DNA technology can be used to produce a locally engineered domain antibody by substitution of the CDR2 of an acceptor anti-TNFα domain antibody (Basran et al. WO 2004/081026; SEQ ID NO: 7; FIG. 3) with a CDR2 from a donor New World primate immunoglobulin.

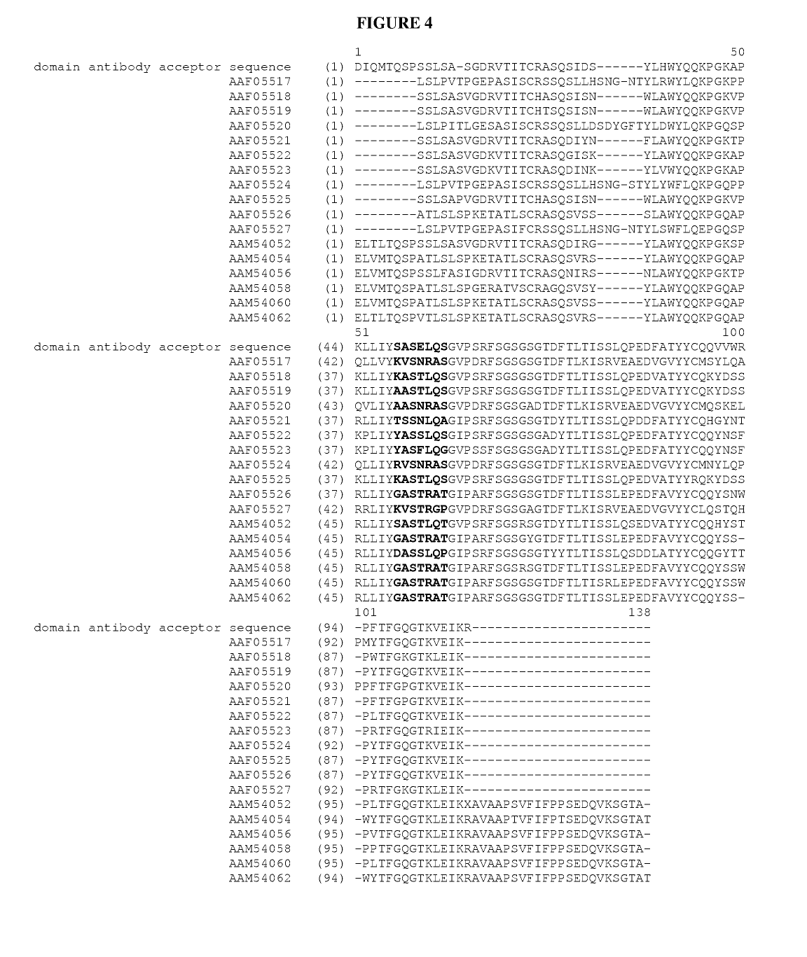

Applying the rules of Kabat (Sequences of Proteins of Immunological Interest” E. Kabat et al., U.S. Department of Health and Human Services, 1983) the CDR2 is identified on the acceptor anti-TNF-α domain antibody (SASELQS (SEQ ID NO.: 39)). The domain antibody acceptor sequence is then aligned against a panel of New World primate immunoglobulin sequences. These sequences are derived from the Ma's night monkey (Aotus nancymaae) (SEQ ID NOs: 8-18) and from the common marmoset (Callithrix jacchus) (SEQ ID NOs: 19-24) (FIG. 4). The CDR2 sequences of the New World primate immunoglobulins that differ from that of the acceptor CDR2 sequence can be identified as SASTLQT (SEQ ID NO.: 40), DASSLQP (SEQ ID NO.: 37), GASTRAT (SEQ ID NO.: 41), KVSNRAS (SEQ ID NO.: 32), RVSNRAS (SEQ ID NO.: 33), KVSTRGP (SEQ ID NO.: 34), AASNRAS (SEQ ID NO.: 35), TSSNLQA (SEQ ID NO.: 36), KASTLQS (SEQ ID NO.: 42), AASTLQS (SEQ ID NO.: 43), YASSLQS (SEQ ID NO.: 44), YASFLQG (SEQ ID NO.: 38) (Table 1). BLAST analysis (http://www.ncbi.nlm.nih.gov/BLAST/) on each of these donor New World primate CDR2 sequences is performed to remove sequences that are exact matches for human immunoglobulin sequences. Sequences unique to New World primates were KVSNRAS (SEQ ID NO.: 32), RVSNRAS (SEQ ID NO.: 33), KVSTRGP (SEQ ID NO.: 34), AASNRAS (SEQ ID NO.: 35), TSSNLQA (SEQ ID NO.: 36), DASSLQP (SEQ ID NO.: 37), YASFLQG (SEQ ID NO.: 38) (Table 1).

TABLE 1

New World primate CDR2 sequences and their
suitability as donor sequences.

| | | Comparison | |
|---|---|---|---|
| SEQ | | to acceptor | BLAST analysis |
| ID | | sequence | against Homo sapien |
| NO | CDR2 sequence | (SASELQS) | sequences |
| 8 | KVSNRAS | Different | No exact matches |
| | (SEQ ID NO.: 32) | | |
| 9 | KASTLQS | Different | Matches human |
| | (SEQ ID NO.: 42) | | |
| 10 | AASTLQS | Different | Matches human |
| | (SEQ ID NO.: 43) | | |
| 11 | AASNRAS | Different | No exact matches |
| | (SEQ ID NO.: 35) | | |
| 12 | TSSNLQA | Different | No exact matches |
| | (SEQ ID NO.: 36) | | |
| 13 | YASSLQS | Different | Matches human |
| | (SEQ ID NO.: 44) | | |
| 14 | YASFLQG | Different | No exact matches |
| | (SEQ ID NO.: 38) | | |
| 15 | RVSNRAS | Different | No exact matches |
| | (SEQ ID NO.: 33) | | |
| 16 | KASTLQS | Different | Matches human |
| | (SEQ ID NO.: 42) | | |
| 17 | GASTRAT | Different | Matches human |
| | (SEQ ID NO.: 41) | | |
| 18 | KVSTRGP | Different | No exact matches |
| | (SEQ ID NO.: 34) | | |
| 19 | SASTLQT | Different | Matches human |
| | (SEQ ID NO.: 40) | | |
| 20 | GASTRAT | Different | Matches human |
| | (SEQ ID NO.: 41) | | |
| 21 | DASSLQP | Different | No exact matches |
| | (SEQ ID NO.: 37) | | |
| 22 | GASTRAT | Different | Matches human |
| | (SEQ ID NO.: 41) | | |
| 23 | GASTRAT | Different | Matches human |
| | (SEQ ID NO.: 41) | | |
| 24 | GASTRAT | Different | Matches human |
| | (SEQ ID NO.: 41) | | |

The acceptor CDR2 and the potential donor CDR2s are examined for their predicted immunogenicity in humans by the MHC class II binding prediction program Propred (http://www.imtech.res.in/raghava/propred) using a 1% threshold value analysis of all alleles. From this analysis the acceptor CDR2, SASELQS (SEQ ID No.: 39), forms part of the peptide, LIYSASELQ (SEQ ID NO.: 45), which is predicted to bind MHC class II encoded by 11 alleles (DRB1_{—}0306, DRB1_{—}0307, DRB1_{—}0308, DRB1_{—}0311, DRB1_0401, DRB1_{—}0426, DRB1_{—}0806, DRB1_{—}0813, DRB1_{—}1501, DRB1_{—}1502, DRB1_{—}1506). The donor CDR2 sequence, KVSNRAS (SEQ ID NO.: 32), forms part of a sequence, LIYKVSNRAS (SEQ ID NO.: 46), which is predicted to bind MHC class II encoded by 9 alleles (DRB1_{—}0309, DRB1_{—}0402, DRB1_{—}0802, DRB1_{—}0804, DRB1_{—}0806, DRB1_{—}0813, DRB1_{—}1301, DRB1_{—}1327, DRB1_{—}1328). The donor CDR2 sequence, AASNRAS (SEQ ID NO: 35), forms part of a sequence, LIYAASNRA (SEQ ID NO.: 47), which is predicted to bind MHC class II encoded by 6 alleles (DRB1_{—}0402, DRB1_{—}0404, DRB1_{—}0408, DRB1_{—}0423, DRB1_{—}0813, DRB1_{—}1506). The donor CDR2 sequence, TSSNLQA (SEQ ID NO.: 36), forms part of a sequence, LIYTSSNLQA (SEQ ID NO.: 48), which is predicted to bind MHC class II encoded by 10 alleles (DRB1_{—}0401, DRB1_{—}0402, DRB1_{—}0404, DRB1_{—}0410, DRB1_{—}0423, DRB1_{—}0426, DRB1_{—}0813, DRB1_{—}1501, DRB1_{—}1502, DRB1_{—}1506). The donor CDR2 sequence, KVSTRGP (SEQ ID NO.: 34), forms part of a sequence LLIYKVSTR (SEQ ID NO.: 49), which is predicted to bind MHC class II encoded by 8 alleles (DRB1_{—}0309, DRB1_{—}0802, DRB1_{—}0804, DRB1_{—}0806, DRB1_{—}0813, DRB1_{—}1301, DRB1_{—}1327, DRB1_{—}1328). Hence, the acceptor CDR2 can be replaced with a donor CDR2 of lower predicted immunogenicity, including KVSNRAS (SEQ ID NO.: 32), AASNRAS (SEQ ID NO.: 35), TSSNLQA (SEQ ID NO.: 36) and KVSTRGP (SEQ ID NO.: 34).

Using recombinant DNA technology, the acceptor CDR2 is replaced with the donor CDR2 sequences, generating the locally engineered domain antibodies (SEQ ID No: 25-31). Examples of recombinant DNA technology include those described by Winter et al. (U.S. Pat. No. 5,225,539), and include, but is not limited to, techniques such as site-directed mutagenesis and oligo annealing. Protein expression of the domain antibodies is then performed in E. coli BL21(DE3) pLys (Novagen, Germany) using a suitable vector for expression such as pET21d(+) (Novagen, Germany), or by other such methods known in the art such as those describe by Basran et al. (WO 2004/081026). Following bacterial cell lysis the domain antibodies are purified using Protein L (Pierce, USA) chromatography.

Following purification the engineered domain antibodies are analysed for retention of TNFα binding ability by methods known in the art, such as the L929 neutralisation assay or the TNFα receptor I binding assay.

To improve the binding affinity of the engineered domain antibodies, affinity maturation could be performed by amino acid substitution of the framework residues surrounding and stabilising CDR2 or by other methods known in the art.(Winter et al. (U.S. Pat. No. 5,225,539); Griffiths et al (U.S. Pat. No. 5,885,793); Rajpal, A. et al. (2005) A general method for greatly improving the affinity of antibodies by using combinatorial libraries, Proc Natl Acad Sci U S A., 102(24) 8466-71; Irving R. A. et al. (2001) Ribosome display and affinity maturation: from antibodies to single V-domains and steps towards cancer therapeutics, Journal of Immunological Methods, 248: 31-45).

### EXAMPLE 3

### Antibodies which Bind TNF-α

### Antibody Cloning

Protein sequences of domain antibodies containing substituted CDR2 sequences (SEQ ID Nos: 25-31) were back-translated into DNA sequences which were optimized for mammalian cell expression using GeneOptimizer technology and synthesized de novo by assembly of synthetic oligonucleotides (GeneArt, Germany). Each gene construct was then restriction digested with Nco I and BamHI/BgIII and ligated into pBAD/gIII (Invitrogen) using the LigaFast Rapid DNA Ligation System from Promega (Cat No. M8221) such that a secretory signal peptide and a 6×HIS tag were introduced into the protein sequence. Ligations were then transformed into One Shot Top 10 (chemically competent cells, Invitrogen, Australia Cat No. C4040-03) and positive colonies identified by standard techniques.

### Expression

A positive colony was selected and grown overday at 37° C. in LB with 50 μg/mL of ampicillin with vigorous shaking. After confirming growth of this colony, a small amount of culture was used to inoculate 10 mL of LB with 50 μg/mL of ampicillin. This culture was grown overnight at 37° C. with vigorous shaking. 500 μL of this culture was used to inoculate 50 mL of LB with 50 μg/mL of ampicillin and the OD of the culture monitored until it reached 0.6. A final concentration of 0.002% L-arabinose (Sigma-Aldrich, Australia) was added to the culture and the induction occurred for 4 hours. The cells were then harvested at 4° C. by centrifugation at 6000 g for 20 mins.

### Purification

The cell pellet was resuspended in osmotic shock solution 1 (20 mM Tris-HCl pH 8.0, 2.5 mM EDTA, 20% sucrose) to an OD of 5.0. The cells were incubated on ice for 10 mins, followed by centrifugation at 6000 g for 10 mins at 4° C. The supernatant was retained and then the cells resuspended in osmotic shock solution 2 (20 mM Tris-HCl pH 8.0, 2.5 mM EDTA) to an OD of 5.0. The cells were incubated on ice for 10 mins, followed by centrifugation at 6000 g for 10 mins at 4° C. The supernatant was kept and pooled with the existing supernatant and then dialysed against binding buffer (20 mM sodium phosphate, pH 7.4, 0.5 M NaCl, 40 mM imidazole [Sigma Aldrich]) overnight with one buffer change. The dialysed sample was then purified on a metal-chelating column (HiTrap HP chelating column, GE Healthcare, Australia) preloaded with NiSO₄. The protein was eluted with elution buffer (20 mM sodium phosphate, pH 7.4, 0.5 M NaCl, 500 mM imidazole) and fractions containing protein were collected, pooled and the sample desalted using Zeba desalting columns (Pierce).

### Anti-TNFα ELISA

TNF-α (Peprotech Cat No: 300-01A) was diluted to 1 μg/mL in carbonate coating buffer (10 mM disodium phosphate, 20 mM sodium hydrogen phosphate pH 9.6). 100 μL of this solution was added to a well of a 96 well plate and incubated at 4° C. overnight in a humidified container. The plate was then washed three times with wash buffer (0.01M PBS pH 7.2, 0.05% Tween-20) and then three times with 0.01M PBS pH 7.2. The wells were then blocked by adding 200 μL blocking buffer (1% w/v BSA in 0.01M PBS pH 7.2) to each well and incubating the plate at 25° C., in a humidified container, for 1 hour. Desalted domain antibody protein sample was diluted in antibody diluent (1% w/v BSA, 0.05% Tween-20 in 0.01M PBS pH 7.2) and added to the wells contain TNF-α and allowed to incubate for 1 hour at 25° C. The plate was then washed as previously described. 100 μL of Anti-HIS antibody HRP conjugate (Sigma-Aldrich, Australia Cat No: A7058) at 1:2000 in antibody diluent was used to detect bound domain antibody. Wells with antibody diluent only were used to assess background absorbance. After incubation at 25° C., in a humidified container, for 1 hour the plate was washed again as previously described. 100 μL TMB substrate solution (Zymed, Cat No: 00-2023) was added to each well and the colour allowed to develop for 4 min. 100 μL of 1M HCl was added to terminate the colour development reaction and absorbance was determined at 450 nm (ref. 620 nm)

### Results

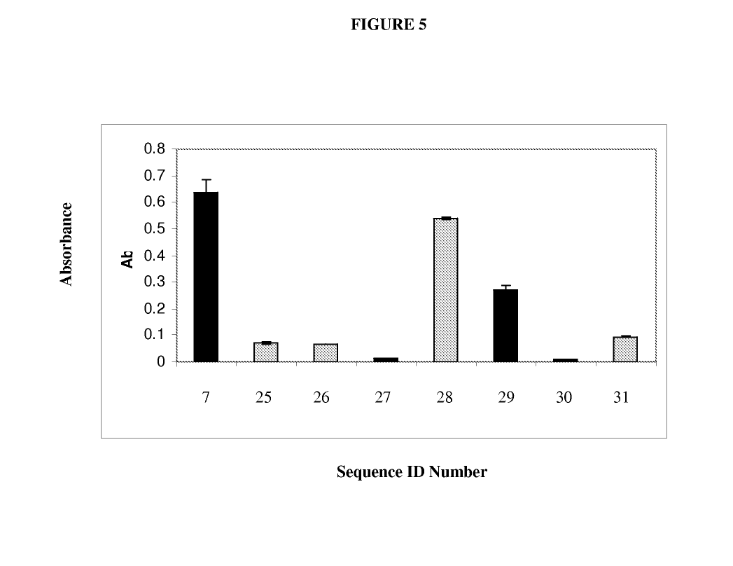

CDR2-substituted domain antibodies encoded by SEQ ID Nos 25, 26, 28, 29 and 31 and the unsubstituted acceptor (SEQ ID No: 7) clearly bound to TNF-α (FIG. 5).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

We claim:

1. A method of producing a chimeric antibody or an antigen-binding portion thereof, the method comprising deleting a CDR from a human antibody variable region comprising at least two CDRs and at least three framework regions and replacing it with a New World primate CDR predicted to be of low immunogenicity to produce a chimeric variable region.

2. The method according to claim 1 wherein the method further comprises the step of recovering the chimeric variable region.

3. The method according to claim 1 wherein the New World primate CDR is CDR2.

4. The method according to claim 1 further comprising the step of modifying the sequence of the chimeric variable region to increase binding, provided that the New World primate CDR sequence is not modified.

5. The method according to claim 1 further comprising the step of modifying the sequence of the chimeric variable region to decrease immunogenicity in humans, provided that the at least one New World primate CDR sequence is not modified.

6. The method according to claim 1 wherein the New World primate is selected from the group consisting of marmosets, tamarins, squirrel monkey, titi monkey, spider monkey, woolly monkey, capuchin, uakaris, sakis, night or owl monkey and the howler monkey.

7. The method according to claim 6 wherein the New World primate is a marmoset.

8. The method according to claim 1 wherein the antibody binds to an antigen that is peptide, protein, carbohydrate, glycoprotein, lipid or glycolipid in nature, selected from a tumour-associated antigen including carcinoembryonic antigen, EpCAM, Lewis-Y, Lewis-Y/b, PMSA, CD20, CD30, CD33, CD38, CD52, CD154, EGF-R, Her-2, TRAIL and VEGF receptors, an antigen involved in an immune or inflammatory disease or disorder including CD3, CD4, CD25, CD40, CD49d, MHC class I, MHC class II, GM-CSF, interferon-γ, IL-1, IL-12, IL-13, IL-23, TNF-α, and IgE, an antigen expressed on a host cell including glycoprotein IIb/IIIa, P-glycoprotein, purinergic receptors and adhesion receptors including CD11a, CD11b, CD11c, CD18, CD56, CD58, CD62 or CD144, an antigen comprising a cytokine, chemokine, growth factor or other soluble physiological modulator or a receptor thereof including eotaxin, IL-6, IL-8, TGF-β, C3a, C5a, VEGF, NGF and their receptors, an antigen involved in central nervous system diseases or disorders including β-amyloid and prions, an antigen of non-human origin such as microbial, nanobial or viral antigens or toxins including respiratory syncitial virus protein F, anthrax toxin, rattle snake venom and digoxin.

9. The method according to claim 8, wherein the antibody binds to TNFα.

10. A chimeric antibody or an antigen-binding portion thereof produced by the method according to claim 1.
